(19) United States
(12) Patent Application Publication
(10) Pub. No.: US 2020/0376206 A1
(43) Pub. Date: Dec. 3, 2020

(54) GAS FEEDING APPARATUS, GAS FEEDING CONTROL METHOD AND STORAGE MEDIUM

(71) Applicant: OLYMPUS CORPORATION, Tokyo (JP)

(72) Inventors: Kunitoshi HIRAGA, Tama-shi (JP); Shinya TORII, Tokyo (JP); Koji YAMAOKA, Hamura-shi (JP); Yuma KASUYA, Hachioji-shi (JP); Takefumi UESUGI, Tachikawa-shi (JP); Keita KIMURA, Hachioji-shi (JP)

(73) Assignee: OLYMPUS CORPORATION, Tokyo (JP)

(21) Appl. No.: 16/932,272

(22) Filed: Jul. 17, 2020

Related U.S. Application Data
(63) Continuation of application No. PCT/JP2018/029098, filed on Aug. 2, 2018.

(30) Foreign Application Priority Data
Jan. 17, 2018 (JP) 2018-005661

(51) Int. Cl. A61M 13/00; G05D 23/19; G05D 7/06

(52) U.S. Cl. CPC A61M 13/003; G05D 23/1931; G05D 7/0623; A61B 1/3132; A61M 2205/3576; A61M 2205/36; A61M 2205/3368

(57) ABSTRACT

A gas feeding apparatus includes first and second temperature sensors for measuring a temperature of an atmosphere that are disposed in a housing. The apparatus may also include a panel, a protrusion protruding forward from the panel, a first opening provided in the panel in a vicinity of the first temperature sensor, and a second opening provided in the panel in a vicinity of the second temperature sensor. The first temperature sensor and the second temperature sensor are separated by a predetermined distance in a horizontal direction and disposed below the protrusion.

### CROSS REFERENCE TO RELATED APPLICATION

This application is a continuation application of PCT/JP2018/029098 filed on Aug. 2, 2018 and claims benefit of Japanese Application No. 2018-005661 filed in Japan on Jan. 17, 2018, the entire contents of which are incorporated herein by this reference.

### BACKGROUND

Conventionally, endoscope systems provided with an endoscope that picks up an image of an object inside a subject and an image processing apparatus that generates an observation image of the object picked up by the endoscope have been widely used in medical and industrial fields. For example, laparoscopic surgical operations, which conduct care and treatment without opening an abdominal cavity for the purpose of minimizing invasion to patients, are practiced in the medical field.

During a laparoscopic surgical operation, a gas feeding apparatus that supplies a feeding gas such as carbon dioxide into the abdominal cavity is used to secure a view of an endoscope and an operation space for a treatment instrument. The gas feeding apparatus is configured to control a decompression valve or a flow rate control valve, adjust the feeding gas to a safe pressure and flow rate, and supply the feeding gas into the abdominal cavity via a gas feeding tube.

The feeding gas is normally supplied to the gas feeding tube at the same temperature as the temperature in an operating room (e.g., 25° C.), but this temperature is a temperature which is lower by about 10° C. than the body temperature (e.g., 37° C.). Therefore, when the feeding gas at the same temperature as the temperature in the operating room is supplied into the body cavity via the gas feeding tube, this may cause a burden on the patient under operation and may induce hypothermia.

Thus, a heater may be housed in the gas feeding tube to heat the feeding gas to be supplied into the body cavity. A temperature sensor may be disposed in the gas feeding tube to determine the temperature of the feeding gas.

In such a configuration, a temperature sensor needs to be disposed in the gas feeding tube, which causes the overall cost of the gas feeding tube to increase. Furthermore, when a temperature sensor or a heater is disposed in the gas feeding tube, the gas feeding tube cannot be cleaned, and so the gas feeding tube needs to be a disposable type gas feeding tube. In this case, a new gas feeding tube is necessary for each procedure, causing a cost increase of the gas feeding tube to directly lead to a cost increase of the procedure.

### SUMMARY

The present disclosure relates to a gas feeding apparatus that supplies a feeding gas such as carbon dioxide into a body cavity or the like, a gas feeding control method, and a storage medium.

A gas feeding apparatus according to one aspect of the present disclosure includes first and second temperature sensors for measuring a temperature of an atmosphere that are disposed in a housing. The apparatus may also include a panel disposed so as to be adjacent to the first temperature sensor and the second temperature sensor, a protrusion protruding forward from the panel, a first opening provided in the panel in a vicinity of the first temperature sensor, and a second opening provided in the panel in a vicinity of the second temperature sensor. The first temperature sensor and the second temperature sensor are separated by a predetermined distance in a horizontal direction and disposed below the protrusion.

A gas feeding control method according to another aspect of the present disclosure acquires a first measurement result measured by a first temperature sensor, acquires a second measurement result measured by a second temperature sensor, calculates a temperature difference between the first measurement result and the second measurement result, and controls a heater or supply of feeding gas according to the calculated temperature difference.

A computer-readable non-transitory recording medium on which a gas feeding control program is stored may be provided according to a further aspect of the present disclosure. The program causes a computer to execute a process of acquiring a first measurement result measured by a first temperature sensor, a process of acquiring a second measurement result measured by a second temperature sensor, a process of calculating a temperature difference between the first measurement result and the second measurement result and a process of controlling a heater or a supply of feeding gas according to the calculated temperature difference.

### BRIEF DESCRIPTION OF THE DRAWINGS

FIG. 1 is a diagram illustrating an example of an overall configuration of a surgical operation system including a gas feeding apparatus according to an exemplary embodiment;

FIG. 2 is a perspective view illustrating an example of an appearance of the gas feeding apparatus;

FIG. 3 is a cross-sectional view illustrating an example of a detailed configuration of an opening portion 15a;

FIG. 4 is a block diagram illustrating an example of a configuration of the gas feeding apparatus;

FIG. 5 is a diagram illustrating an example where a heat source is placed in the vicinity of the gas feeding apparatus;

FIG. 6 is a diagram illustrating an example of a relationship between a distance from the heat source and a detected temperature;

FIG. 7 is a diagram illustrating another example where a heat source is placed in the vicinity of the gas feeding apparatus;

FIG. 8 is a flowchart illustrating an example of a flow of abnormality detection processing;

FIG. 9 is a diagram illustrating a configuration of a gas feeding apparatus according to an exemplary embodiment; and

FIG. 10 is a diagram illustrating a configuration of a gas feeding apparatus according to an exemplary embodiment.

### DETAILED DESCRIPTION OF THE PREFERRED EMBODIMENTS

Hereinafter, embodiments of the present disclosure will be described with reference to the accompanying drawings.

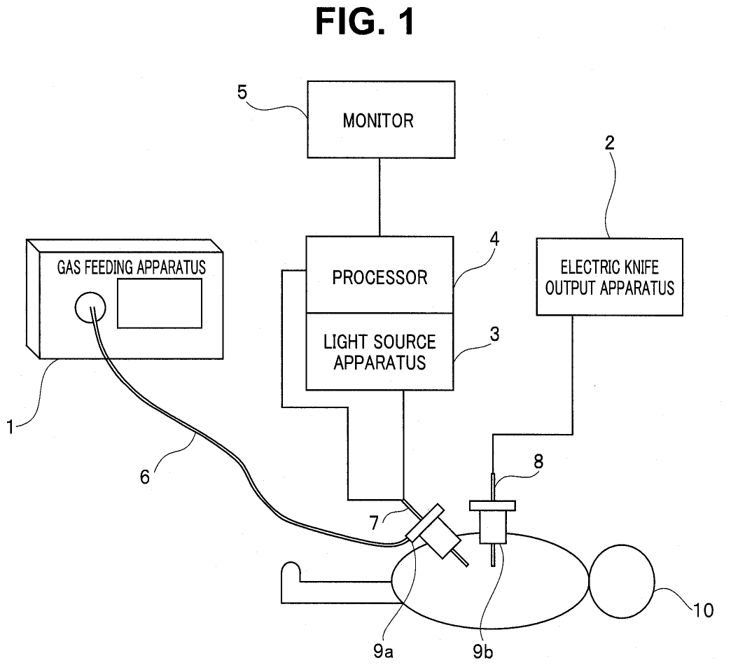

FIG. 1 is a diagram illustrating an example of an overall configuration of a surgical operation system including a gas feeding apparatus according to an exemplary embodiment.

As shown in FIG. 1, a surgical operation system of the present embodiment is used for a surgical operation treating an affected part in the abdominal cavity of a patient 10 expanded by feeding a gas such as carbon dioxide under observation of an endoscope 7 using a treatment instrument such as an electric knife 8.

As shown in FIG. 1, a first trocar 9a and a second trocar 9b are punctured on an abdominal wall of the patient 10. The first trocar 9a is a trocar for guiding the endoscope 7 into the abdominal cavity. A gas feeding tube 6, which will be described later, is connected to the first trocar 9a and configured to guide a feeding gas such as carbon dioxide supplied from the gas feeding apparatus 1 into the abdominal cavity. The second trocar 9b is a trocar for guiding a treatment instrument such as the electric knife 8 that performs excision or treatment of tissue into the abdominal cavity.

A light source apparatus 3 and a processor 4 are connected to the endoscope 7. A monitor 5 is connected to the processor 4. The light source apparatus 3 guides light emitted from the semiconductor light source using a light guide member, converts a color, luminous intensity distribution or the like using an optical conversion member provided at a distal end of the light guide member and supplies illumination light to the endoscope 7. The processor 4 supplies a supply voltage to the endoscope 7, applies predetermined video signal processing to the image pickup signal picked up by the endoscope 7 and outputs a video signal to the monitor 5. Thus, an endoscope image (surgical image) obtained by the endoscope 7 is displayed on the monitor 5.

An electric knife output apparatus 2 is connected to the electric knife 8. The electric knife output apparatus 2 outputs a high-frequency current to the electric knife 8 to generate high-frequency electrical energy. By bringing an electrode at a distal end of the electric knife 8 into contact with tissue of an affected part of the patient 10, the high-frequency current outputted from the electric knife output apparatus 2 is intensively passed to the tissue of the affected part to generate Joule heat and perform dissection of the tissue of the affected part or hemostatic coagulation at a bleeding site.

A cylinder (not shown) filled with carbon dioxide (CO₂ gas) is connected to the gas feeding apparatus 1 that feeds the feeding gas. Furthermore, one end of the gas feeding tube 6 is connected to the gas feeding apparatus 1. The other end of the gas feeding tube 6 is connected to the first trocar 9a punctured on the abdominal wall of the patient 10. That is, the gas feeding apparatus 1 is enabled to feed the feeding gas such as carbon dioxide into the abdominal cavity of the patient 10 via the gas feeding tube 6 and the first trocar 9a.

The aforementioned gas feeding apparatus 1, the electric knife output apparatus 2, the light source apparatus 3, the processor 4 and the monitor 5 are mounted, for example, on a movable trolley apparatus. Furthermore, the configuration of the surgical operation system is not limited to the configuration in FIG. 1, but may also have a configuration including a circulating smoke exhaust apparatus. The circulating smoke exhaust apparatus is configured to aspirate carbon dioxide gas containing smoke or the like generated by use of the electric knife 8 from within the abdominal cavity of the patient 10, remove the smoke or mist from the aspirated carbon dioxide gas, and then return the carbon dioxide gas into the abdominal cavity.

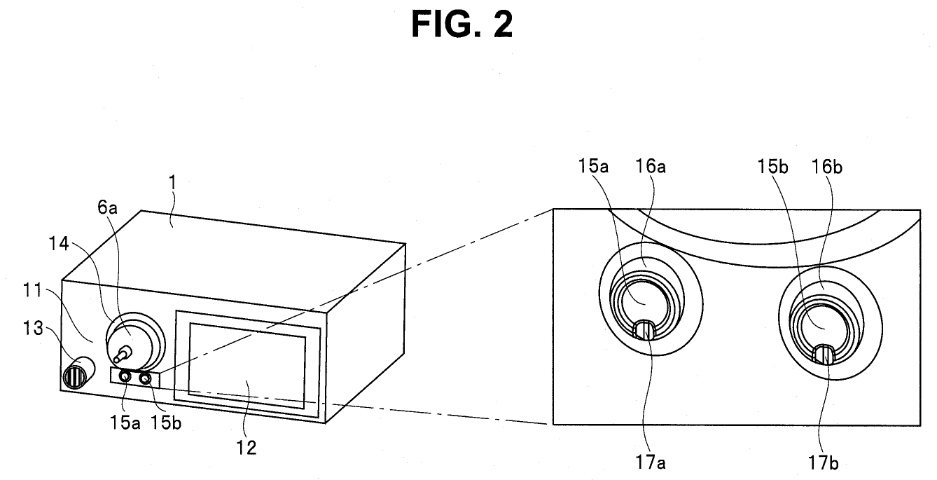

Next, the configuration of the gas feeding apparatus 1 of the present embodiment will be described using FIG. 2 to FIG. 4.

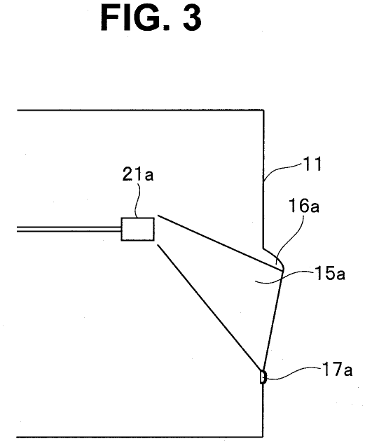

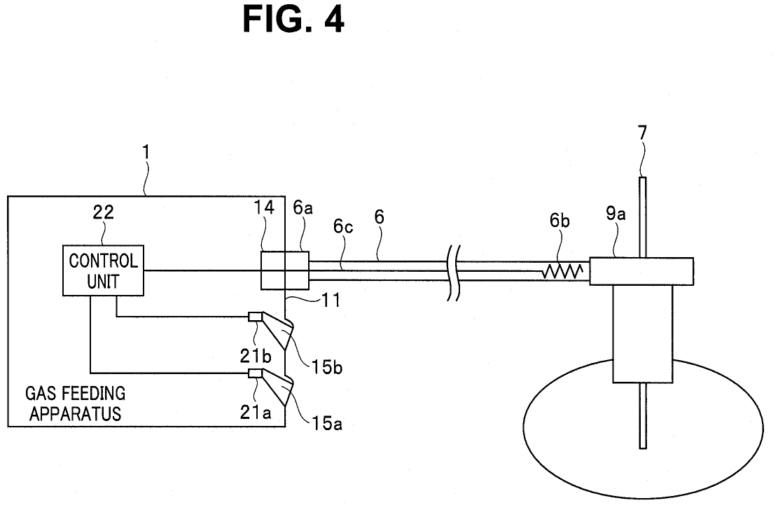

FIG. 2 is a perspective view illustrating an example of an appearance of the gas feeding apparatus, FIG. 3 is a cross-sectional view illustrating an example of a detailed configuration of an opening portion 15a and FIG. 4 is a block diagram illustrating an example of a configuration of the gas feeding apparatus.

As shown in FIG. 2, the gas feeding apparatus 1 is configured to include a panel part 11 as a front panel and a display unit 12 on the front. The panel part 11 is disposed so as to be adjacent to first and second temperature sensors 21a and 21b, which will be described later, and configured to measure a temperature of the atmosphere disposed inside (in the housing) of the gas feeding apparatus 1. Furthermore, the panel part 11 is provided with a pinch valve 13, a gas feeding connector receiving part 14 and two opening portions 15a and 15b.

The two opening portions 15a and 15b are provided separated by a predetermined distance in the horizontal direction and below the gas feeding connector receiving part 14. Note that the gas feeding apparatus 1 is configured to include the two opening portions 15a and 15b and the two temperature sensors 21a and 21b, but without being limited to this, the gas feeding apparatus 1 may have a configuration including three or more opening portions and three or more temperature sensors.

A gas feeding tube connector 6a (see FIG. 4) provided at a proximal end portion of the gas feeding tube 6, which will be described later, is connected to the gas feeding connector receiving part 14. Note that the gas feeding tube 6 is not illustrated in FIG. 2.

Protrusion-shaped umbrella parts 16a and 16b are provided around the opening portions 15a and 15b so as to prevent a liquid from falling over the opening portions 15a and 15b, and the first and second temperature sensors 21a and 21b, which will be described later. Furthermore, slits 17a and 17b are provided from lower surface parts of the opening portions 15a and 15b toward the umbrella parts 16a and 16b disposed around. The slits 17a and 17b are slits to guide liquid films, which may be generated in the opening portions 15a and 15b, to the outside.

As shown in FIG. 3, the opening portion 15a is provided so as to have a predetermined angle diagonally upward with respect to the panel part 11 as the panel. Furthermore, the opening portion 15a is tapered in such a way as to broaden toward the bottom from the top side (the proximal end side where the temperature sensor 21a is provided) to the bottom side (the distal end side where the panel part 11 is provided). Note that although the opening portion 15a will be described as an example in FIG. 3, the opening portion 15b has also a similar configuration.

The first temperature sensor 21a is provided on the proximal end side of the opening portion (first opening) 15a. A second temperature sensor 21b is provided on the proximal end side of the opening portion (second opening) 15b as shown in FIG. 4. Note that in FIG. 4, although the opening portions 15a and 15b are disposed in the vertical direction for simplicity of description, it is assumed that the opening portions 15a and 15b are disposed in the horizontal direction as shown in FIG. 2.

The first temperature sensor 21a and the second temperature sensor 21b are configured to contact the atmosphere through the opening portion 15a and the opening portion 15b, and measure a room temperature. The measurement results measured by the first temperature sensor 21a and the second temperature sensor 21b are outputted to a control unit 22, which is provided in the gas feeding apparatus 1 and will be described later.

In this way, the first temperature sensor 21a and the second temperature sensor 21b are provided on the proximal end side of the opening portions 15a and 15b respectively. As described above, the opening portions 15a and 15b are separated by a predetermined distance in the horizontal direction and provided below the gas feeding connector receiving part 14, and so the first temperature sensor 21a and the second temperature sensor 21b are also separated by a predetermined distance in the horizontal direction and disposed below the gas feeding connector receiving part 14.

As shown in FIG. 4, the gas feeding apparatus 1 is configured to include the control unit 22 in addition to the aforementioned panel part 11, the gas feeding connector receiving part 14, the opening portions 15a and 15b, the first temperature sensor 21a, the second temperature sensor 21b and so on.

The gas feeding tube connector 6a is provided at the proximal end portion of the gas feeding tube 6. The gas feeding tube connector 6a is connected to the gas feeding connector receiving part 14 of the gas feeding apparatus 1.

A heater 6b for heating the feeding gas is disposed on the distal end side inside the gas feeding tube 6. The heater 6b is connected to a cable 6c passed through the gas feeding tube 6. The cable 6c is configured such that it is connected to the control unit 22 when the gas feeding tube 6 is connected to the gas feeding apparatus 1. The heater 6b is configured to heat the feeding gas in the gas feeding tube 6 to a constant temperature zone close to the body temperature (e.g., 35 to 39° C.).

The first temperature sensor 21a measures the room temperature via the opening portion 15a and outputs the first measurement result to the control unit 22. The second temperature sensor 21b measures the room temperature via the opening portion 15b and outputs the second measurement result to the control unit 22.

The control unit 22 as a processor calculates a temperature difference between the first measurement result measured by the first temperature sensor 21a and the second measurement result measured by the second temperature sensor 21b and detects whether the temperature difference is equal to or greater than a predetermined value. This predetermined value is assumed to be, for example, 1° C. in consideration of respective errors of the first temperature sensor 21a and the second temperature sensor 21b (e.g., variations during manufacturing and errors in measurement). Note that all or some functions of the plurality of circuits of the processor may be executed by software. For example, the processor including hardware may be constructed of a central processing unit (CPU), a ROM and a RAM so that the CPU reads and executes various programs corresponding to the respective functions stored in the ROM.

When the control unit 22 detects that the temperature difference does not exceed the predetermined value, the control unit 22 determines that the room temperature has been measured correctly. The control unit 22 adjusts a heating amount of the heater 6b, that is, a current value to be passed to the heater 6b based on the first and second measurement results (room temperature) measured by the first and second temperature sensors 21a and 21b. More specifically, the control unit 22 adjusts a current value to be passed to the heater 6b based on an average value of the first and second measurement results. The control unit 22 is configured to heat the heater 6b in the gas feeding tube 6 to thereby heat the feeding gas flowing through the gas feeding tube 6 and supply the feeding gas at substantially the same temperature as a body temperature of the patient 10 into the abdominal cavity of the patient 10. For example, the feeding gas may be heated to a temperature within a range of from 35 to 39° C.

On the other hand, when the control unit 22 detects that the temperature difference exceeds the predetermined value, the control unit 22 determines that the room temperature has not been measured correctly. When the control unit 22 detects that the room temperature has not been measured correctly, that is, the control unit 22 detects that an abnormality has been detected, the control unit 22 stops heating the feeding gas and stops a supply of the feeding gas into the abdominal cavity of the patient 10.

When the control unit 22 detects that an abnormality has occurred, the control unit 22 notifies the user that the abnormality has occurred. The control unit 22, for example, displays on the display unit 12 that the abnormality has been detected, and thereby notifies the user of the abnormality. Note that the notification of an abnormality is not limited to a display on the display unit 12 that the abnormality has been detected, but an LED (not shown) or the like may be turned on or a warning sound may be outputted from a speaker (not shown).

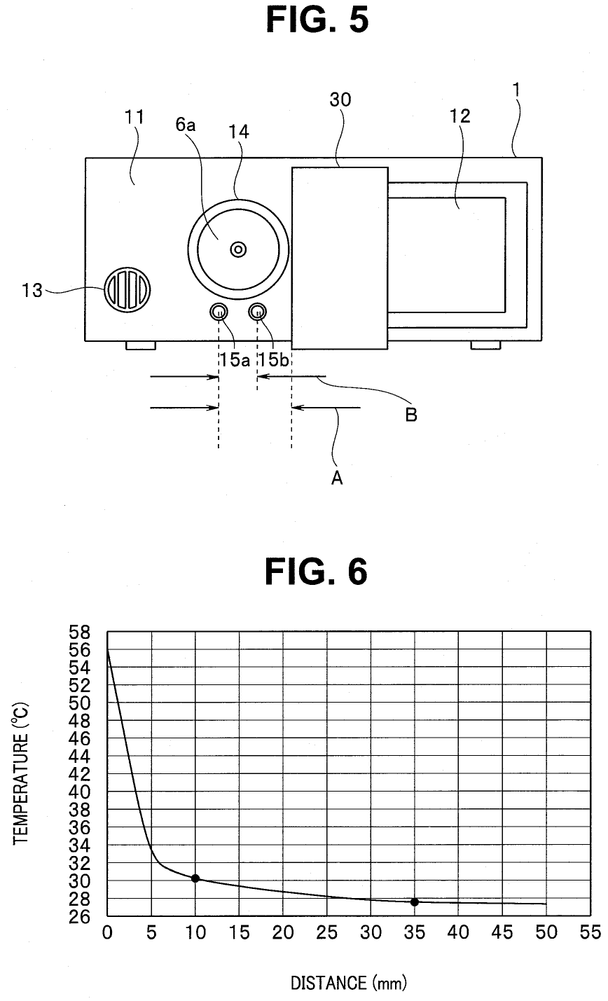

FIG. 5 is a diagram illustrating an example where a heat source is placed in the vicinity of the gas feeding apparatus and FIG. 6 is a diagram illustrating an example of a relationship between a distance from the heat source and a detected temperature.

As shown in FIG. 5, a heat source 30, which becomes a disturbance, may be placed in the vicinity of the gas feeding apparatus 1. The heat source 30 is, for example, hot water for cleaning a lens surface provided on the distal end face of the insertion portion of the endoscope 7.

The gas feeding tube connector 6a is connected to the gas feeding connector receiving part 14 provided on the panel part 11, constituting a protrusion. When the protrusion exists on the panel part 11, the heat source 30 is placed so as to avoid the protrusion, and so the heat source 30 is placed on the left side or the right side of the protrusion. Note that in the example in FIG. 5, the heat source 30 is placed on the right side of the protrusion when viewed from the front of FIG. 5.

In this case, a distance A between the heat source 30 and the first temperature sensor 21a disposed on the proximal end side of the opening portion 15a is, for example, 50 mm (>35 mm).

As shown in FIG. 6, when the distance from the heat source 30 is 35 mm or more, the temperature measured is substantially constant and is not affected by the heat source 30. For this reason, the first temperature sensor 21a can measure the room temperature correctly without being affected by the heat source 30.

A distance B between the first temperature sensor 21a and the second temperature sensor 21b is, for example, 28 mm (>25 mm). As shown in FIG. 6, when two measuring points (e.g., first and second temperature sensors 21a and 21b) are separated apart by 25 mm or more as in the case where the distances between: (i) the first and second temperature sensors 21a and 21b and (ii) the heat source 30 are 10 mm and 35 mm, respectively, the temperature measured by the second temperature sensor 21b is increased by 2° C. or more under the influence of the heat source 30 as compared to the first temperature sensor 21a.

Since the second temperature sensor 21b is disposed more than 25 mm away from the first temperature sensor 21a, the measured value is higher than the measurement result of the first temperature sensor 21a by 2° C. or more due to the influence of the heat source 30. Therefore, the second temperature sensor 21b cannot accurately measure the room temperature under the influence of the heat source 30.

As a result, the control unit 22 recognizes that a temperature difference between the first measurement result of the first temperature sensor 21a and the second measurement result of the second temperature sensor 21b is equal to or greater than the predetermined value, and can detect an abnormal state in which the room temperature cannot be measured accurately under the influence of the heat source 30.

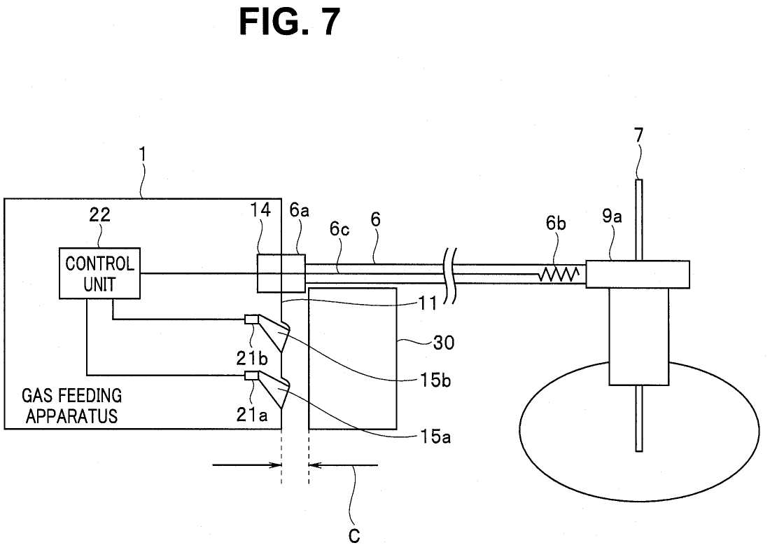

Note that a case has been described in the example in FIG. 5 where the heat source 30 is placed on the right side of the protrusion, but without being limited to this, the heat source 30 may be placed in front of the protrusion. FIG. 7 is a diagram illustrating another example where the heat source is placed in the vicinity of the gas feeding apparatus.

As shown in FIG. 7, the gas feeding tube connector 6a, which is a protrusion, has such a protruding length that a distance C between the heat source 30 and the opening portion 15a or 15b for temperature measurement becomes a predetermined distance (35 mm) or more. Therefore, even when the heat source 30 is placed in front of the protrusion, or more specifically, in front of the opening portions 15a and 15b, the distance of the opening portion 15a or 15b from the heat source 30 becomes 35 mm or more. As a result, the first temperature sensor 21a and the second temperature sensor 21b can correctly measure the room temperature without being affected by the heat source 30.

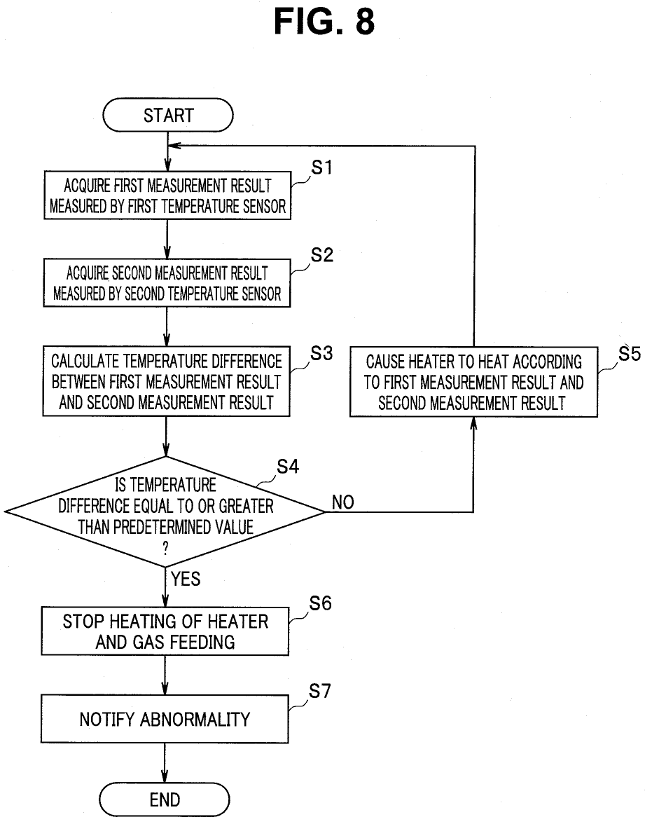

FIG. 8 is a flowchart illustrating an example of a flow of abnormality detection processing. The processing in FIG. 8 is performed by the control unit 22 deploying and executing a gas feeding control program stored in the ROM over the RAM.

The control unit 22 acquires the first measurement result measured by the first temperature sensor 21a (S1) and acquires the second measurement result measured by the second temperature sensor 21b (S2). Next, the control unit 22 calculates a temperature difference between the first measurement result and the second measurement result (S3) and determines whether the temperature difference is equal to or greater than a predetermined value (S4). Here, the control unit 22 determines whether the temperature difference is equal to or greater than 1° C.

When the control unit 22 determines that the temperature difference is not equal to or greater than a predetermined value (S4: NO), the control unit 22 causes the heater 6b to heat according to the first measurement result and the second measurement result (S5) and returns to the process in S1. On the other hand, when the control unit 22 determines that the temperature difference is equal to or greater than the predetermined value (S4: YES), the control unit 22 stops heating and gas feeding of the heater 6b (S6). Finally, the control unit 22 notifies the abnormality (S7) and ends the process. The control unit 22 notifies the user of the abnormality, for example, by causing the display unit 12 to display that the abnormality has been detected.

As described above, the gas feeding apparatus 1 provides two opening portions 15a and 15b of the panel part 11 on the front contacting the atmosphere and provides two temperature sensors (first and second temperature sensors 21a and 21b) on the proximal end side of the opening portions 15a and 15b. In this way, the gas feeding apparatus 1 prevents a room temperature from being erroneously measured under the influence of a disturbance by an air-conditioner or the like.

The gas feeding apparatus 1 causes the first temperature sensor 21a and the second temperature sensor 21b to be disposed separated by a predetermined distance in the horizontal direction below the protrusion of the panel part 11. In this way, when the heat source 30 is placed so as to avoid the protrusion, a temperature difference is generated between the temperature measured by the first temperature sensor 21a and the temperature measured by the second temperature sensor 21b, and the gas feeding apparatus 1 can detect an abnormal state under the influence of a disturbance by the heat source 30 or the like.

Thus, according to the gas feeding apparatus 1 of the present embodiment, it is possible to prevent the temperature sensor disposed on the apparatus from erroneously measuring the room temperature under the influence of a disturbance.

Next, another exemplary embodiment will be described.

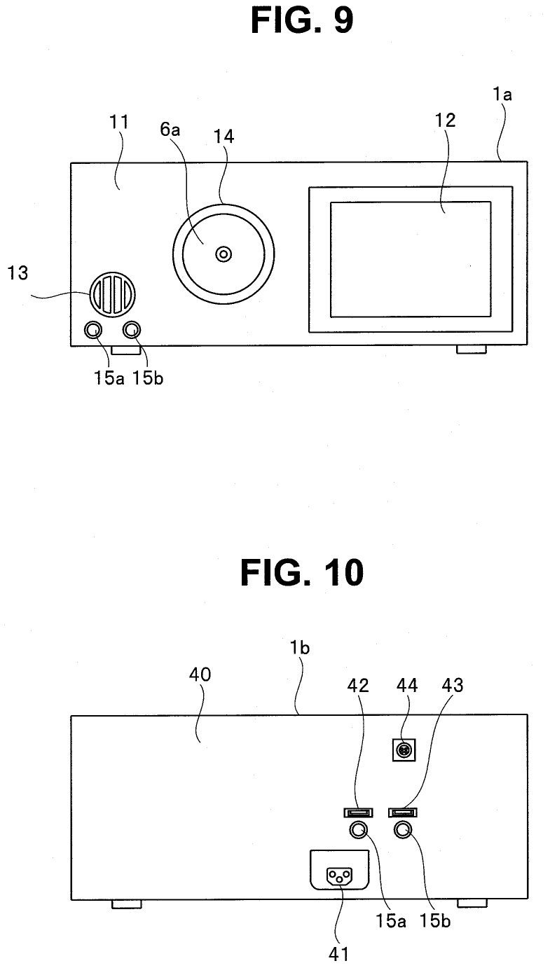

FIG. 9 is a diagram illustrating a configuration of a gas feeding apparatus according to another exemplary embodiment. Note that in FIG. 9, components similar to the components in FIG. 2 are assigned the same reference numerals and description is omitted.

In the aforementioned embodiment, the gas feeding tube connector 6a has been described as an example of a protrusion provided on the panel part 11, but the present disclosure is not limited to this.

As shown in FIG. 9, a gas feeding apparatus 1a of the present embodiment is provided with the opening portions 15a and 15b separated by a predetermined distance in the horizontal direction below the pinch valve 13 provided on the panel part 11. In the present embodiment, the pinch valve 13 provided on the panel part 11 constitutes a protrusion.

As in the case of the above embodiment, the first temperature sensor 21a and the second temperature sensor 21b are disposed on the proximal end side of the opening portions 15a and 15b respectively. The other components are similar to the components of the above embodiment.

As described so far, the gas feeding apparatus 1a of the present embodiment can prevent the temperature sensor disposed in the apparatus from erroneously measuring the room temperature under the influence of a disturbance as in the case of the above embodiment.

Next, another exemplary embodiment will be described.

FIG. 10 is a diagram illustrating a configuration of a gas feeding apparatus according to the present embodiment.

As shown in FIG. 10, a rear panel 40 is provided on a rear surface of a gas feeding apparatus 1b. A power connector 41, and a plurality of electrical connectors 42, 43 and 44 are disposed on the rear panel 40.

The gas feeding apparatus 1b of the present embodiment is provided with the opening portions 15a and 15b separated by a predetermined distance in the horizontal direction below the electrical connectors 42 and 43 provided on the rear panel 40. In the present embodiment, the electrical connectors 42 and 43 constitute protrusions.

The first temperature sensor 21a and the second temperature sensor 21b are disposed on the proximal end side of the opening portions 15a and 15b respectively as in the case of the above embodiment. The other components are similar to the components of the above embodiment.

As described so far, the gas feeding apparatus 1b of the present embodiment can prevent the temperature sensor disposed in the apparatus from erroneously measuring the room temperature under the influence of a disturbance as in the case of the above embodiment.

Note that an arrangement of the opening portions 15a and 15b is not limited to the arrangement in FIG. 10. In the gas feeding apparatus 1b, for example, the opening portions 15a and 15b may be disposed separated by a predetermined distance in the vertical direction. That is, the gas feeding apparatus 1b may have a configuration in which the opening portion 15a is disposed below an electrical connector 44 and the opening portion 15b is disposed below an electrical connector 43.

In the gas feeding apparatus 1b, the opening portions 15a and 15b may be disposed separated diagonally by a predetermined distance. That is, the gas feeding apparatus 1b may have a configuration in which the opening portion 15a is disposed below the electrical connector 42 and the opening portion 15b is disposed below the electrical connector 44.

Note that the respective steps in the flowchart in the present specification may be executed in a different order of execution or a plurality of steps may be executed simultaneously or may be executed in order differing every time as long as it is not contrary to the nature of the specification.

The present disclosure is not limited to the aforementioned embodiments but various changes, modifications or the like can be made without departing from the gist of the disclosure.

What is claimed is:

1. A gas feeding apparatus comprising:
a first temperature sensor disposed in a housing and configured to measure a temperature of an atmosphere;
a second temperature sensor disposed in the housing and configured to measure a temperature of the atmosphere;
a panel disposed so as to be adjacent to the first temperature sensor and the second temperature sensor;
a protrusion protruding forward from the panel;
a first opening provided in the panel in a vicinity of the first temperature sensor; and
a second opening provided in the panel in a vicinity of the second temperature sensor,
wherein the first temperature sensor and the second temperature sensor are separated by a predetermined distance in a horizontal direction and are disposed below the protrusion in a vertical direction transverse to the horizontal direction.

2. The gas feeding apparatus according to claim 1, wherein the first opening and the second opening are disposed below the protrusion in the vertical direction.

3. The gas feeding apparatus according to claim 1, wherein the protrusion has a protruding length such that a disturbance placed in front of the first opening and the second opening is blocked by the protrusion from being disposed at a distance less than a predetermined distance from the first opening and the second opening.

4. The gas feeding apparatus according to claim 1, further comprising a processor configured to stop a supply of feeding gas when a temperature difference between a temperature measured by the first temperature sensor and a temperature measured by the second temperature sensor is determined to be equal to or greater than a predetermined value.

5. The gas feeding apparatus according to claim 4, wherein the processor is further configured to:
calculate the temperature difference between the temperature measured by the first temperature sensor and the temperature measured by the second temperature sensor, and
determine whether the temperature difference is equal to or greater than the predetermined value.

6. The gas feeding apparatus according to claim 4, wherein the processor is configured to notify a user of an abnormality when the temperature difference is determined to be equal to or greater than the predetermined value.

7. The gas feeding apparatus according to claim 1, wherein the protrusion is any one of a pinch valve, a gas feeding tube connector and an electrical connector.

8. The gas feeding apparatus according to claim 1, wherein the panel is a rear panel, and the first and the second temperature sensors are provided inside the rear panel.

9. The gas feeding apparatus according to claim 1, wherein the horizontal direction and the vertical direction are orthogonal to a protruding direction of the protrusion.

10. The gas feeding apparatus according to claim 1, wherein the first temperature sensor is disposed inside the first opening, and the second temperature sensor is disposed inside the second opening.

11. The gas feeding apparatus according to claim 10, wherein the first temperature sensor is disposed inside the first opening at a position spaced from an outer surface of the panel from which the protrusion protrudes, and the second temperature sensor is disposed inside the second opening at a position spaced from the outer surface of the panel.

12. The gas feeding apparatus according to claim 10, wherein the first opening and the second opening extend in a depth direction of the gas feeding apparatus from an outer surface of the panel to the first temperature sensor and the second temperature sensor, respectively, so as to be inclined in a direction towards the protrusion.

13. A gas feeding control method comprising:
acquiring a first measurement result measured by a first temperature sensor;
acquiring a second measurement result measured by a second temperature sensor;
calculating a temperature difference between the first measurement result and the second measurement result; and
controlling a heater or a supply of feeding gas according to the calculated temperature difference.

14. The gas feeding control method according to claim 13, wherein the heater is controlled by adjusting a current value transmitted to the heater based on an average value of the first measurement result and the second measurement result.

15. The gas feeding control method according to claim 13, wherein the heater is controlled by adjusting a current value transmitted to the heater such that the feeding gas is supplied into a cavity of a patient at substantially the same temperature as a body temperature of the patient.

16. The gas feeding control method according to claim 13, wherein the supply of feeding gas is controlled by stopping the supply of feeding gas when the temperature difference between the first measurement result and the second measurement result is determined to be equal to or greater than a predetermined value.

17. A computer-readable non-transitory recording medium with an executable gas feeding control program stored thereon, the program causing a computer to execute:
a process of acquiring a first measurement result measured by a first temperature sensor;
a process of acquiring a second measurement result measured by a second temperature sensor;
a process of calculating a temperature difference between the first measurement result and the second measurement result; and
a process of controlling a heater or a supply of feeding gas according to the calculated temperature difference.
